(19) United States
(12) Patent Application Publication
(10) Pub. No.: US 2010/0035286 A1
(43) Pub. Date: Feb. 11, 2010

(54) IN VITRO METHOD FOR THE DIAGNOSIS AND EARLY DIAGNOSIS OF NEURODEGENERATIVE DISORDERS

(75) Inventors: Andreas Bergmann, Berlin (DE); Andrea Ernst, Hennigsdorf (DE); Harald Hampel, Munchen (DE)

Correspondence Address: HESLIN ROTHENBERG FARLEY & MESITI PC, 5 COLUMBIA CIRCLE, ALBANY, NY, 12203, US

(73) Assignee: B.R.A.H.M.S AKTIENGESELLSCHAFT, Hennigsdorf (DE)

(21) Appl. No.: 12/516,851

(22) PCT Filed: Nov. 28, 2007
(86) PCT No.: PCT/EP2007/010331

§ 371 Date: Aug. 19, 2009

(30) Foreign Application Priority Data
Nov. 29, 2006 (DE) 102006056337.9

(51) Int. Cl. G01N 33/535; G01N 33/92

(52) U.S. Cl. 435/7.94; 436/71

(57) ABSTRACT

In vitro method for the detection and early detection of, for the determination of the degree of severity of, and for the assessment of the course of and prognosis of neurodegenerative disorders, in which an immunodiagnostic determination method is used to determine the apolipoprotein C-1 (Apo C-1) immunoreactivity in a serum or plasma sample from a patient suffering from subjective or objectively verifiable cognitive disorders.

The present invention relates to a novel in vitro method for the diagnosis and in particular early diagnosis of neurodegenerative disorders, in particular of dementias, such as Alzheimer's disease and precursors thereof.

In the context of the present invention, the term “diagnosis” is used as an overall term for medical determinations which may be based on different problems according to the clinical condition of the patient for whom the determination is carried out and which serve for the detection and, in the present case, in particular the early detection, the determination of the severity and the assessment of the course, including the therapy-accompanying assessment of the course, and the prognosis of the future course of a disease. What is of particular importance in the present context is that a diagnosis may also be a negative diagnosis in which the presence of a certain disease is made improbable, for example owing to the nondetectability of a certain biomarker concentration typical of the disease in a blood sample of a patient.

Also of considerable value for the negative diagnosis are biomarkers for which pathologically changed concentrations can be found in the case of a plurality of different diseases and which by themselves therefore still do not permit a positive diagnosis of a specific disease—although as a rule they may also be decisive for the positive diagnosis on inclusion of further clinical or biochemical parameters

The diseases regarding the diagnosis of which the present invention is concerned tend to be slowly developing, chronic neurodegenerative disorders of non-infectious etiology, in particular dementias.

Dementias are generally designated as diseases for which a common feature is the impairment of acquired intellectual abilities, especially of memory, and of the normal personality level as a result of brain damage. Dementias are as a rule relatively slowly developing diseases of a chronic character. If dementias occur prior to old age, in middle age, they are referred to as presenile dementias. Dementias are differentiated on the basis of the symptoms and cerebropathological changes typical of them, in particular the following diseases or groups of diseases:

Alzheimer's disease (AD) is the most frequent neurodegenerative dementia, accounts for ⅔ of all cases of dementia and is also the practically most important field of use for the present invention. AD is distinguished by three important pathological features which however can be detected only post-mortem: the formation of amyloid plaques and neurofibrillar bundles and the loss of nerve cells (for an overview cf (1); literature references in the description in the form of numbers relate to the list of references following the description). Amyloid plaques consist of extraneuronal aggregates of the amyloid-β protein, while the neurofibrillar bundles contain mainly tau-protein and neurofilaments. It is presumed that the plaque and neurofibril formation is the cause of the death of nerve cells.

The most important symptoms of AD are increasing impairment of the capacity to register and disturbance of intellectual function in combination with relatively persistent emotional responsiveness, these symptoms being accompanied by further less specific disturbances which make it more difficult to distinguish AD from other forms of dementia.

Observations of AD patients and patients who developed AD in the course of their clinical observation over many years led to the formulation of criteria for mutually distinguishable groups of patients which cover the entire range of
a. persons without subjective and objective cognitive impairments (which in the context of the present application represent the control group) through
b. patients who complain about subjective diminished cognitive ability but in whom no cognitive deficits can be found (in the context of the present invention, this is the group of “SCI” patients, where “SCI” represents “subjective cognitive impairments”), further through
c. patients who have been found to have mild cognitive impairments and who have been diagnosed with “possible AD” (“pos AD”) where no other dementia-causing diseases are present (in the context of the present application, this is the group “MCI pos AD”, where “MCI” represents “mild cognitive impairments”) to
d. the group of patients with the typical clinical picture for considerable cognitive impairments which have begun gradually and progress slowly, which patients are diagnosed with “probable AD” when other causes of dementia can be ruled out (in the context of the present application, this is the group “pr AD”, where the abbreviation represents “probable Alzheimer's”).

Regarding the assignment of patients with subjective and/or objective cognitive impairments to various groups, reference is additionally made to (2), (3), (4), (5).

Dementia with lewy bodies (DLB) is the second most frequent cause of a dementia after Alzheimer's disease. Neuropathologically, DLB is characterized by the occurrence of so-called lewy bodies in the brain stem and in the cortex. These lewy bodies predominantly comprise aggregates of the presynaptic protein α-synuclein and ubiquitin. The lewy body pathology can be associated to various extents with Alzheimer- and Parkinson-typical neuropathological changes. Thus, in the case of DLB too, the formation of beta-amyloid and senile plaques occurs, but not neurofibril bundles (for an overview, cf (6)). Lewy bodies are also present in the brain of patients with Parkinson's disease, although in a different distribution.

The key symptoms of DLB are a progressive cognitive impairment, episodes of confusion with fluctuating attentiveness and awareness, Parkinsonism, frequent falls and syncopes (brief, paroxysmal unconsciousness). The sensitivity and specificity of the diagnostic criteria show a high specificity throughout but in some cases a very low sensitivity. This means that DLB is frequently not diagnosed in day-to-day clinical routine.

Frontotemporal dementia (FTD) is also referred to as Pick's disease and accounts for about 20% of presenile dementias. FTD is in some cases of genetic origin and is among the so-called tauopathies, which are distinguished by an overexpression or underexpression of a tau-protein subtype or by the expression of a mutated tau-protein. Neuropathologically, local atrophy of the frontal and/or temporal cortex and of the substantia nigra and of the basal ganglia occurs. This results in speech disturbances of varying severity, a personality change and behavioural abnormalities. Overall, FTD is underdiagnosed with a sensitivity of 93% and a specificity of only 23%, AD representing the most frequent misdiagnosis.

The term vascular dementia (VAD) summarizes diseases in which a dementia is triggered by blood flow disturbances in the brain. There are different types of VAD, of which multi-infarction dementia (MID) and subcortical VAD (also referred to as Binswanger's disease) are the most frequent forms.

Binswanger's disease is a slowly progressing dementia which is characterized pathologically by cerebrovascular lesions in the white brain substance. Clinically, this results in behavioural abnormalities, such as agitation, irritability, depression and euphoria, and slightly impaired memory.

Multi-infarction dementia occurs gradually as a result of a plurality of small strokes, also referred to as transient ischemic attacks (TIA), which led to the destruction of brain tissue in the cortex and/or subcortical areas. The strokes may also have remained completely unnoticed, in which case the dementia is the first noticeable consequence. When MID is present, there is a gradual decrease in cognitive abilities, associated with severe depressions, mood variations and epilepsy.

A diagnosis of dementias is carried out nowadays predominantly on the basis of neuropsychological investigations and observation of the development of the disease and its course, using exclusion criteria for certain forms of dementia. In very many cases, these investigations give ambiguous results, which explain the abovementioned numbers for the underdiagnosed forms of dementia or incorrectly diagnosed cases. The cerebral changes typical of the disease cannot of course be determined directly on living patients, and investigations of the brain functions using medical equipment by means of, for example, computer-aided tomography or magnetic resonance imaging are complicated and expensive.

It would therefore be desirable to supplement and thereby considerably improve the detection and in particular early detection of dementias by the measurement of informative biomarkers which can be determined with the aid of a relatively simple test method, for example in a blood sample (serum sample, plasma sample) of a patient.

For the diagnosis of Alzheimer's disease, the Ronald and Nancy Regan Institute of the Alzheimer's Association and the NIA Working Group published guidelines for the criteria which are set regarding an ideal biomarker for the detection of AD (7). The following criteria should ideally be fulfilled by the biomarker:
1. It should be brain-specific and detect a fundamental feature of the neuropathology of these diseases.
2. The diagnostic sensitivity and the specificity of at least 80% should exist.
3. The disease-specific change of the biomarker should manifest itself in as early a stage as possible of the disease, in order to be able to begin suitable therapeutic measures (8).

Up to the present, however, there is no biomarker which is detectable in the circulation (i.e. in the blood or in blood preparations, such as serum or plasma) of a patient and which could be used in day-to-day clinical routine for improving the early diagnosis and differential diagnosis of AD and fulfils all abovementioned criteria. At present, various potential marker candidates are being investigated, including inflammation markers, such as IL-6 and TNFα, markers for oxidative stress, such as 3-nitrotyrosene, and markers which are associated with characteristic pathological changes of AD, such as amyloid β which is a main constituent of the amyloid plaques, and the tau-protein, which is a substantial constituent of the neurofibril bundles (cf the overview in (7); (9)).

There is a current demand for supplementary investigative methods which provide valid laboratory findings and which are based on a determination of substances suitable as biomarkers for dementias, in particular for Alzheimer's disease (AD), in blood or plasma samples and are suitable for supporting an early positive diagnosis and/or for a negative diagnosis by exclusion in the case of patients who are suspected of having a dementia, in particular AD.

The present invention provides such an investigative method in the form of an in vitro method for the detection and early detection, for the determination of the severity and for the assessment of the course and prognosis of neurodegenerative disorders, in which the apo C-I immunoreactivity is determined in a serum or plasma sample of a patient who is suffering from subjective or objectively detectable cognitive impairments, with the aid of an immunodiagnostic assay method, and in which conclusions regarding the presence of a neurodegenerative disorder or of an early form typical of this or regarding the course of the disorder and/or the success of the efforts for its alleviation or prevention are drawn on the basis of the concentration measured therefor.

Advantageous or preferred configurations of the method according to claim 1 are reproduced in subclaims 2 to 8.

The basis of the present invention is the experimental finding that reduced concentrations of the immunoreactive apo C-I are measured in a characteristic manner in the blood (serum, plasma) of patients with cognitive impairments, including those diagnosed with “possible Alzheimer's” or “probable Alzheimer's” in comparison with control persons.

Where the term “immunoreactive apo C-I” or “immunodiagnostically determinable apo C-I” is used in the context of the present application, it defines an analyte as can be determined with the aid of an immunoassay, in particular an immunoassay as described in more detail in the experimental section of the present application or in the German Patent Application DE 103 43 815 A1 of the applicant.

As explained in said DE 103 43 815 A1—the entire content of which, where this relates to the assay procedure and the discussion on the apo C-I molecules or apo C-I derivatives determinable therewith, is referred to expressly for supplementing the disclosure of the present application—an immunoassay of said type is used to determine, in blood of healthy control persons, those apo C-I fractions which have a capability of binding to hydrophobic molecular structures or hydrophobic surfaces and which can be removed from the sample by reaction with a suitable material for the hydrophobic exclusion chromatography (e.g. octyl sepharose).

The apo C-I which can be determined in the manner described or the corresponding apo C-I derivative is also designated as “free apolipoprotein C-I”. It has the properties of being bound from a serum or plasma sample (in PBS dilution) to hydrophobic molecular structures, e.g. the octyl radicals of a hydrophobic octyl sepharose chromatography material, from which it can be eluted under typical conditions for the elution of proteins, in particular with diluted acetic acid. The use of the term “free apolipoprotein C-I”, however, does not necessarily mean that the material separated off by hydrophobic interaction chromatography must be present completely freely in the original samples. Associates or aggregates, also with lipids, which are cleaved under the experimental conditions on contact with octyl sepharose with formation of the apolipoprotein C-I on the chromatography material are likewise to be regarded as “free apolipoprotein C-I” in the context of the use of this term.

As described in said DE 103 43 815 A1, said immunoassay detects in the blood of cancer patients also an apo C-I fraction which does not bind to octyl sepharose and whose occurrence appears to be characteristic for tumour diseases. If appropriate, in the overall diagnosis for a dementia or for the precursors thereof using the measured values obtained in the determination of immunoreactive apo C-I, it would therefore be necessary to rule out that the measured values are falsified in the case of the respective patient by a cancer. This can be effected, for example, in the manner described in DE 103 43 815 A1 by checking whether the measured concentration values change through contact of the sample with, for example, octyl sepharose.

The immunoreactive apo C-I to be determined according to the present invention comprises, in addition to analytes which represent or contain the complete peptide apo C-I, also “apo C-I derivatives”. These are to be understood as meaning in particular immunoreactive apo C-I fragments and aggregates, in particular those which behave like the free protein apolipoprotein C-I in said sandwich immunoassay. The “derivatives” may be, for example, apolipoprotein C-I molecules shortened by individual amino acids or amino acid sequences, or complete apolipoprotein C-I molecules stearically or conformationally modified—for example by aggregation.

That the invention is also to comprise the determination of such apo C-I derivatives is taken into account by the known facts that immunoassays normally cannot distinguish between different analytes when the differences are outside the molecular segments used for the antibody binding, i.e. in the case of a sandwich immunoassay, in end regions of the determined molecule outside the amino acid sequence bounded by the two binding sites for the antibodies. Furthermore, a further known fact to be taken into account is that, when peptidic analytes occur in the circulation, the occurrence of their degradation products in the form of peptide fragments should also always be expected. The concentrations of such fragments are as a rule approximately proportional to the concentration of the starting peptide, the concentration differences reflecting stability differences or different speeds of “clearing” of the peptides or fragment by further proteolytic degradation or by elimination from the circulation, for example via the kidneys.

Where a discussion of apo C-I in association with Alzheimer's disease can be found in the literature, it relates exclusively to investigations of the apolipoprotein C-I expression in extracts of brain tissue of dead persons, but not in the circulation of living patients (cf 10). In view of the existence of the blood-brain barrier (BBB), which exerts a barrier effect for the passage of, for example, protein substances between the brain and the circulation, no conclusions regarding the occurrence or the concentration of protein substances in the circulation can be drawn from investigations with brain tissue extracts.

Where fractions from sera of two Alzheimer's patients were also measured in the course of the chromatographic investigations described in DE 103 43 815 A1, no statistically significant results were obtained which would permit a conclusion regarding a relationship between the concentration of the immunoreactive apo C-I and various stages of dementias or of Alzheimer's disease.

The measured results in EDTA plasma samples of 60 apparently healthy normal persons (symptom-free controls) and 196 patients with mild to severe cognitive impairments according to the groups (b) to (d) described at the outset are described below in the experimental section and gave for the first time a clear, diagnostically significant correlation between the concentrations found for immunoreactive apo C-I and the severity of the dementia symptoms in the form of cognitive impairments, the measured concentrations correlating in a significant manner with the severity of AD precursors and thus reflecting the clinical differentiation of the various patient groups.

The determination, according to the invention, of immunoreactive apo C-I in the diagnosis of neurodegenerative disorders is proposed in particular as a measurement which supplements the parallel determination of physiological or clinical and neuropsychological parameters and other biomarkers and refines said determination for differential diagnostic purposes.

The method is therefore preferably carried out as part of a multiparameter determination in which at least one further biochemical or physiological parameter informative for the respective clinical picture is simultaneously determined and in which a measured result in the form of a set of at least two measured variables, which is evaluated for the fine diagnosis of the neurodegenerative disorder, is obtained.

In addition to the determination of the immunoreactive apo C-I, for example, it is possible to determine at least one further biochemical parameter which is selected from the groups consisting of the natriuretic peptides, inflammation mediators, complement components, cytokines, chemokines, the blood coagulants and fibrinolytic factors, acute phase proteins and free radical compounds. Co-parameters which may also be mentioned in particular are those parameters which are discussed in the prior German Patent Applications of the applicant with the application numbers 10 2005 034 174.8 (mid-regional proadrenomedullin), 10 2005 036 094.7 (liquor diagnostic determination of procalcitonin), 10 2006 027 818.6 and 10 2006 023 175.9 and the parameter CPS-1 (carbamoyl phosphate synthetase 1), about which relevant discussions with regard to neurodegenerative disorders appear in the applications EP 05023420.2 and DE 10 2006 021 406.4.

It is intended that the method according to the invention can also be effected as a simultaneous determination by means of a chip technology measuring apparatus or an immunochromatographic measuring apparatus and the evaluation of any complex measured results obtained thereby can be effected with the aid of a suitable computer program.

Although the investigations are limited to date to plasma samples of patients who showed signs of precursors of AD or who have been diagnosed with “probable Alzheimer's”, the inventors assume that—possibly with different typical concentration ranges—characteristic changes of the concentrations of the immunoreactive apo C-I could be found in patient plasmas also in the case of other neurodegenerative disorders, in particular in vascular dementia (VAD) and dementia with lewy bodies (DLB).

Below, the invention is explained in more detail with reference to measured results and a FIGURE.

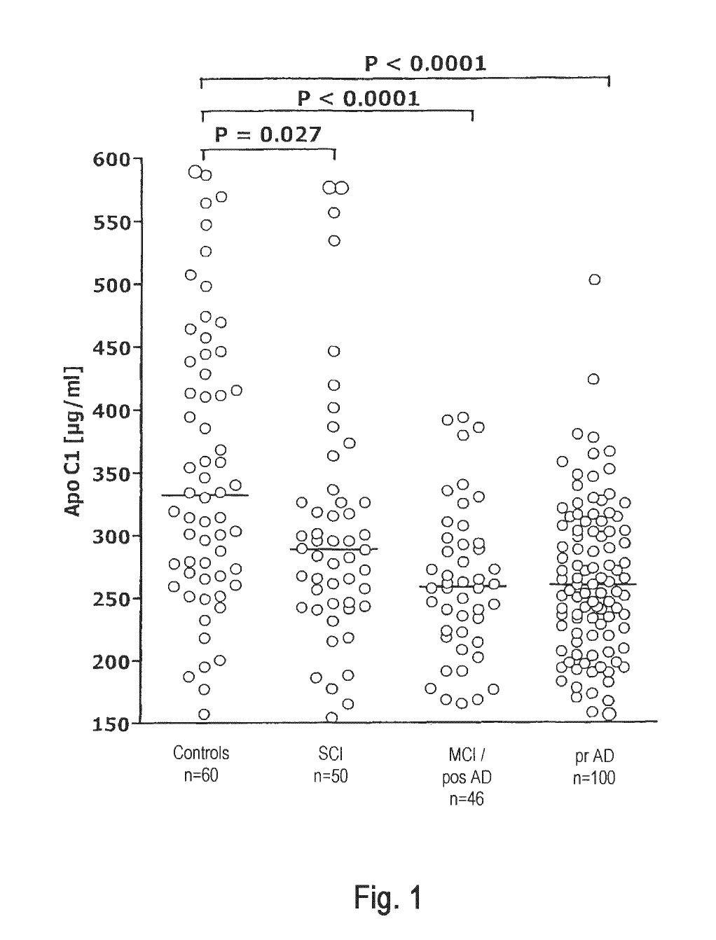

FIG. 1 shows the results of the measurement of the concentrations of the immunoreactive apo C-I as can be determined by the sandwich immunoassay described in the experimental section, in particular in EDTA plasmas of 60 healthy control persons and of 196 patients with cognitive impairments of various severity, who corresponded to the abovementioned groups b), c) and d), i.e. the groups “SCI” (50 patients), “NCI pos AD” (46 patients) and “prob AD” (100 patients).

### Experimental Section

1. Apolipoprotein C-I Immunoassay

The direct measurement of immunodiagnostically determinable apo C-I in plasma was effected using the same sandwich immunoassay as that described in the German Patent Application DE 103 43 815 A1 of the applicant. Specifically, this assay has the following structure:

For the direct immunodiagnostic determination of apolipoprotein C-I in serum, an immunoassay of the sandwich type was set up from the constituents described below:
a. Coated tubes: polystyrene tubes (Greiner) were coated with a commercially available polyclonal affinity-purified antibody against apolipoprotein C-I (source; Acris antibody A, Bad Neuheim, Germany). According to the manufacturer's information, the antibody had been obtained by immunisation of rabbits with human apo C-I and had been purified via a sepharose affinity column with human apolipoprotein C-I. For the coating, 0.2 μg of antibody in 300 μl of PBS was bound to polystyrene tubes (Greiner, Germany) which were coated with sheep anti-rabbit IgG antibodies (Sigma). The binding was complete after 18 hours at room temperature. The tubes were then washed twice with 3 ml portions of 0.5% bovine serum albumin (BSA) in PBS. After they had been tried in vacuo, the tubes were used as a solid phase for the apolipoprotein C-I immunoassay.
b. Acridinium ester-marked antibody: 100 μg of another antibody against human apolipoprotein C-I (from rabbit; source: Academie Bio-Medical Company, Texas, USA) in 100 μl of PBS were reacted with 10 μg of acridinium NHS ester (in 10 μl of acetonitryl). After incubation for 10 minutes at room temperature, the purification of the marked antibody was effected, with separation of unconverted constituents of the reaction mixture by HPLC on SW 300 (Waters). For its use in the immunoassay, the marked antibody in PBS was adjusted with 0.5% of BSA and 1 mg/ml of rabbit IgG for saturation of the tube walls to about 1 million RLU/300 μl (RLU=relative light units).

Carrying Out the Immunodiagnostic Determination of Apolipoprotein C-I in Serum or Plasma Samples

Serum or plasma samples were diluted 1:10 000 with PBS, 0.5% BSA. In each case 300 μl thereof were pipetted into the abovementioned tubes coated with the immobilized antibody and were then incubated for 4 hours at room temperature with shaking (300 rpm on a Heidolph rotary shaking machine). The tube content was then washed out with PBS (filling and decanting 4 times with 1 ml of PBS in each case) and apolipoprotein C-I bound to the tube wall was reacted with 300 μl per tube of the acridinium ester-marked anti-apolipoprotein C-I antibody within 20 hours at room temperature and 300 rpm. Thereafter, unbound marked antibody was removed by washing 5 times with 1 ml of PBS each time, and the remaining chemiluminescence was measured in a known manner in a Berthold 952 T illuminometer.

For calibration of the assay, a synthetic apolipoprotein C-I was used. A typical standard curve, as obtained for the above assay, is shown in FIG. 3 of the abovementioned DE 103 43 815 A1.

Measurement of the Apo C-I Immunoreactivity in the Plasma of Healthy Controls in Patients with Cognitive Impairments of Different Severity

For determining a reference value for the concentration of the immunodiagnostically determined “free” apo C-I, a measurement was carried out in EDTA plasmas of 60 symptom-free control persons who neither showed symptoms of cognitive impairments nor suffered from any other detectable disease (tumour diseases; severe infection or inflammation) which are known to influence the apo C-I level in the circulation. For the control group, a median value of 332 μg/ml was determined from the measured apo C-I concentration.

Patients with dementia symptoms in the form of cognitive impairments of various severity, on the basis of which the individual patients were assigned to one of the abovementioned groups as b), c) or d), served as the patient group.

The measured concentrations of immunoreactive apo C-I in the plasma of healthy controls and patients with cognitive impairments are shown in FIG. 1

The determined numerical values in the form of the so-called medians for the various patient groups were as follows:

| | Median | Median | Median | Median |
|---|---|---|---|---|
| | Control | SCI | MCI pos AD | prob AD |
| | (n = 60) | (n = 50) | (n = 46) | (n = 100) |
| apo C-I (data | 332.9 | 288.5 | 258.5 | 260.0 |
| μg/ml) | | | | |

The concentrations of the apo C-I which can be determined immunodiagnostically in plasmas, evident from the values for the medians of the various patient groups, clearly decrease with the severity of the symptoms in the direction:
controls>SCI>MCI pos AD≈prob AD

From the abovementioned prior DE 103 43 815 A1, it is known that the measurable concentrations of apo C-I show deviations from the values measurable in control persons even in the case of other diseases or physiological conditions; however, most relevant diseases, such as, for example, sepsis or tumours, can as a rule easily be clinically differentiated diagnostically from dementias and their possible effects can therefore be taken into account appropriately when making a diagnosis.

Although apo C-I is therefore not a brain-specific parameter, the measurement of the apo C-I immunoreactivity in the circulation (plasma, serum) is very suitable for the purposes of supportive early diagnosis of AD, in view of the abovementioned correlations.

### LITERATURE REFERENCES

1. SELKOE D. J. (2001). Alzheimer's disease: genes, proteins, and therapy. Physiological Reviews 81: 741-766
2. Boetsch T., Stütbner S. Auer S., Klinisches Bild, Verlauf und Prognose, Chapter 5 in: Hampel, Padberg, Möller (editors), Alzheimer Demenz—Klinische Verläufe, diagnostische Möglichkeiten, moderne Therapiestrategien; WVG mbH Stuttgart 2003
3. Boetsch T., Operationalisierte Demenzdiagnostik, Chapter 6.1 in: Hampel, Padberg, Möller (editors), Alzheimer Demenz—Klinische Verläufe, diagnostische Möglichkeiten, moderne Therapiestrategien; WVG mbH Stuttgart 2003
4. Reisberg B., Ferris S. H., de Leon M. J., Crook T., 1982, The global deterioration scale for assessment of primary degenerative dementia, Am J Psychiatry 139:1136-1139
5. McKhann G., Drachmann D., Folstein M., Katzman R., Price D., Stadlan E. M. 1984, Clinical diagnosis of Alzheimer's disease: Report of the NINCDS-ARDA work group under the auspices of department of health services task force on Alzheimer's disease, Neurology 24: 939-944
6. MCKEITH I. G. (2002). Dementia with lewy bodies. British Journal of Psychiatry 180: 144-147
7. FRANK R. A., GALASKO D., HAMPEL H., HARDY J., DE LEON M. J., MEHTA P. D., ROGERS J., SIEMERS E., TROJANOWSKI J. Q. (2003). Biological markers for therapeutic trials in Alzheimer's disease. Proceedings of the biological markers working group; NIA initiative on neuroimaging in Alzheimer's disease. Neurobiology of Aging 24: 521-536
8. GROWDON J. H., SELKOE D. J., ROSES A., TROJANOWSKI J. Q., DAVIES P., APPEL S. et al. [Working Group Advisory Committee]. (1998). Consensus report of the Working Group on Biological Markers of Alzheimer's Disease. [Ronald und Nancy Reagan Institute of the Alzheimer's Association and National Institute on Aging Working Group on Biological Biomarkers of Alzheimer's Disease]. Neurobiology of Aging 19: 109-116
9. TEUNISSEN C. E., DE VENTE J., STEINBUSCH H. W. M., DE BRUIJN C. (2002). Biochemical markers related to Alzheimer's dementia in serum and cerebrospinal fluid. Neurobiology of Aging 23:485-508
10. C. PETIT-TURCOTTE, S. M. STOHL, U. BEFFERT, J. S. COHN, N. AUMONT, M. TREMBLAY, D. DEA, LIN YANG, J. POIRIER, and N. S. SHACHTER (2001). Apolipoprotein C-I Expression in the Brain in Alzheimer's Disease, Neurobiology of Disease 8, 953-963.

1. A method for the detection and early detection, for the determination of the severity and for the assessment of the course and prognosis of neurodegenerative disorders, in which the concentration of the immunoreactive apolipoprotein apo C-I is determined in a serum or plasma sample of a patient who is suffering from subjective or objectively detectable cognitive impairments with the aid of an immunodiagnostic assay method, and in which conclusions regarding the presence of a neurodegenerative disorder or of an early form thereof typical of this or regarding the course of the disorder and/or the success of the efforts for its alleviation or treatment are drawn on the basis of the measured concentration.

2. The method of claim 1, wherein the immunodiagnostic assay method is an immunoassay of the sandwich type.

3. The method of claim 1, wherein the neurodegenerative disorder is a dementia selected from the group consisting of Alzheimer's disease (AD), dementia with lewy bodies (DLB), frontotemporal dementia (FTD) and various forms of vascular dementia (VD).

4. The method of claim 4, wherein said method is carried out as part of Alzheimer diagnostics for detecting early forms of Alzheimer's disease (AD).

5. The method of claim 1, wherein said method is carried out as part of a multiparameter determination in which at least one further biochemical or physiological parameter informative for the respective clinical picture is simultaneously determined and in which a measured result in the form of a set of at least two measured variables, which is evaluated for fine diagnosis of the neurodegenerative disorder, is obtained.

6. The method of claim 5, wherein, in addition to the determination of the immunoreactive apo C-I, at least one further biochemical parameter selected from the groups consisting of the natriuretic peptides, inflammation mediators, complement components, cytokines, chemokines, the blood coagulants and fibrinolytic factors, acute phase proteins and free radical compounds is determined as part of the multiparameter determination.

7. The method of claim 5, wherein the multiparameter determination is effected as a simultaneous determination by means of a chip technology measuring apparatus or by means of an immunochromatographic measuring apparatus.

8. The method of claim 5, wherein the evaluation of the complex measured result of the multiparameter determination is effected with the aid of a computer program.

9. A method for the detection of dementia in a human patient in whom cognitive ability is diminished, said method comprising:
(a) obtaining a blood, plasma, or serum sample from said patient; and
(b) determining the amount of human apolipoprotein (apo C-I) in said sample using an immunodiagnostic assay, wherein a decreased level of apo C-I compared to normal controls indicates dementia.
